SEQUENCE LISTING (12 sequences)

SEQ ID NO: 1 - protein, 381 aa, Cellulomonas sp.
MSEVPVRTLISPLRYVQARGALTRLGEFVRPIGTKPLLVADDVVWGIVQDTVAVSFQEQDLPRHRTGFGTYATAAEVDRLAREIDELGADVIVGIGGGSTIDAVKAAGHLRGIRWVSVPTVASTDAPCSALSVIYTEDGAFEEYRFFPHNPDLVLVDTGLVANAPVKFLVAGVGDALATWIEARAVAEANASTMAGGLPLVTGTALAQLSWDILWENALPAIDAVKNHLVTPAVEKVVEANTLLSGLGFESGGLAAAHAIHNGLTAAPQTHGLTHGQKVNIGSVTQLVLEGAPTEEIEEFVRFTTRVGLPNTLTEIGLTADDVHDLTRVAEAATAEGETIHAMPFPVRVPELVDALRSIEGFSRRVRAEAGLPEPVKHTAH

SEQ ID NO: 2 - DNA, 1146 bp, Cellulomonas sp.
  CDS (1)..(1143)
atgtccgaggttcccgtccgcacgctcatcagcccgctgcgctacgtccaggcccgcggtgcgctcacccgcctcggcgagttcgtccggccgatcggcacgaagccgctgctcgtcgccgacgacgtcgtgtggggcatcgtccaggacaccgtcgccgtgtccttccaggagcaggacctcccccgccaccgcaccggcttcggcacgtacgcgaccgccgccgaggtcgaccggctcgcgcgcgagatcgacgagctcggcgctgacgtgatcgtcggcatcggcggcggctcgaccatcgacgcggtcaaggccgcgggccacctgcgcggcatccgctgggtctccgtgccgaccgtcgcgtccaccgacgccccgtgctccgcgctgtccgtcatctacaccgaggacggcgccttcgaggagtaccggttcttcccgcacaacccggacctcgtgctcgtcgacacgggcctcgtcgcgaacgcgcccgtgaagttcctcgtcgccggcgtcggtgacgcgctcgcgacgtggatcgaggcgcgcgccgtcgccgaggcgaacgcgtcgaccatggccggtggcctccccctcgtcacgggcacggcgctcgcgcagctctcgtgggacatcctctgggagaacgcgctgcccgcgatcgacgcggtgaagaaccacctcgtgacgcccgcggtcgagaaggtcgtcgaggcgaacacgctcctgtccgggctgggcttcgagtccggcggcctcgcggcggcgcacgccatccacaacggcctcaccgccgcgccccagacgcacgggctcacgcacggccagaaggtgaacatcggctccgtgacgcagctcgtgctggagggcgcgccgaccgaggagatcgaggagttcgtgcgcttcacgacgcgcgtcggcctgccgaacacgctcaccgagatcggcctgaccgccgacgacgtgcacgacctcacgcgcgtcgcggaagccgccaccgcggagggcgagacgatccacgccatgccgttccccgtgcgcgtgcccgagctcgtggacgcgctgcgctccatcgagggcttttcccgccgcgtccgcgccgaggccggcctccccgagccggtcaagcacaccgcccactga

SEQ ID NO: 3 - DNA, 20 bp, Artificial Sequence
  PCR primer-1
acngaygarggngmnttyga

SEQ ID NO: 4 - DNA, 20 bp, Artificial Sequence
  PCR primer-2
ggcatntkrtgdatngtytc

SEQ ID NO: 5 - DNA, 24 bp, Artificial Sequence
  PCR primer-3
ttctcccagaggatgtcccacgag

SEQ ID NO: 6 - DNA, 24 bp, Artificial Sequence
  PCR primer-4
agatcgaggagttcgtgcgcttca

SEQ ID NO: 7 - DNA, 27 bp, Artificial Sequence
  PCR primer-5
gatcatatgtccgaggttcccgtccgc

SEQ ID NO: 8 - DNA, 33 bp, Artificial Sequence
  PCR primer-6
ctagaattcttatcagtgggcggtgtgcttgac

SEQ ID NO: 9 - DNA, 43 bp, Artificial Sequence
  PCR primer-7
gccgaattctaaggaggttaacaatgtccgaggttcccgtccg

SEQ ID NO: 10 - DNA, 32 bp, Artificial Sequence
  PCR primer-8
gcgggatccttatcagtgggcggtgtgcttga

SEQ ID NO: 11 - DNA, 43 bp, Artificial Sequence
  PCR primer-9
gccggatcctaaggaggttaacaatgtataaadatttagaagg

SEQ ID NO: 12 - DNA, 29 bp, Artificial Sequence
  PCR primer-10
gcgctgcagttatccgcgtcctgcttgga

(19) United States
(12) Patent Application Publication
(10) Pub. No.: US 2008/0305534 A1
(43) Pub. Date: Dec. 11, 2008

(54) Novel Glycerol Dehydrogenase, Gene Therefor, and Method of Utilizing the Same

(76) Inventors: Daisuke Moriyama, Hyogo (JP); Naoaki Taoka, Hyogo (JP)

Correspondence Address: SUGHRUE MION, PLLC, 2100 PENNSYLVANIA AVENUE, N.W., SUITE 800, WASHINGTON, DC, 20037, US

(21) Appl. No.: 11/629,181

(22) PCT Filed: Jun. 15, 2005
(86) PCT No.: PCT/JP2005/010960

§ 371 Date: Dec. 11, 2006

(30) Foreign Application Priority Data
Jun. 21, 2004 (JP) 2004-182926

(51) Int. Cl. C12P 7/04; C12N 9/02; C12N 15/11; C12N 15/00; C12N 1/20

(52) U.S. Cl. 435/157; 435/189; 536/23.2; 435/320.1; 435/252.33

(57) ABSTRACT

The present invention provides a polypeptide having physicochemical characteristics of (1) to (5) described below:
(1) action: to generate (S)-3-chloro-1,2-propanediol by stereoselectively reducing 1-chloro-3-hydroxyacetone using NADH as a coenzyme;
(2) molecular weight: about 340,000 by gel-filtration and about 43,000 by SDS polyacrylamide gel electrophoresis;
(3) optimum temperature: from 60 to 70° C.;
(4) optimum pH for reduction: 6.0; and
(5) optimum pH for oxidation: 9.0.

Furthermore, the present invention provides a polypeptide comprising the amino acid sequence represented by SEQ ID NO: 1 in the Sequence Listing, a DNA encoding the polypeptide, and a transformant producing the polypeptide in large quantities. Still furthermore, the present invention provides a manufacturing method by using the above polypeptide or the above transformant of (S)-3-chloro-1,2-propanediol that is a useful material for pharmaceuticals, etc.

### TECHNICAL FIELD

The present invention relates to a novel polypeptide, a gene encoding the polypeptide, a vector comprising the gene, a transformant comprising the vector, and a manufacturing method of optically active alcohols by utilizing the transformant.

More specifically, the present invention relates to glycerol dehydrogenase that is isolated from microorganisms having an enzyme activity to stereoselectively reduce 1-chloro-3-hydroxyacetone to generate (S)-3-chloro-1,2-propanediol and that has the above enzyme activity; a DNA encoding the enzyme; an expression vector comprising the DNA; and a transformant transformed by the expression vector. The present invention also relates to a manufacturing method of (S)-3-chloro-1,2-propanediol that is a useful intermediate for pharmaceuticals, etc.

Glycerol dehydrogenase is an enzyme that catalyzes a reaction generating dihydroxyacetone from glycerol using oxidized nicotinamide adenine dinucleotide (herein after referred to as NAD+) or oxidized nicotinamide adenine dinucleotide phosphate (herein after referred to as NADP) as a coenzyme thereof, and a reaction stereoselectively reducing a carbonyl group of α-hydroxyketones using reduced nicotinamide adenine dinucleotide (herein after referred to as NADH) or reduced nicotinamide adenine dinucleotide phosphate (herein after referred to as NADPH) as a coenzyme thereof.

Glycerol dehydrogenase, which not only can be utilized for quantitative determination of glycerol, etc., but also can be used as a catalyst for production of optically active 1,2-diols that are useful as raw materials for synthesis of pharmaceuticals and the like, is an industrially useful enzyme.

### BACKGROUND ART

Glycerol dehydrogenase is known to be present in green algae, yeasts, molds, bacteria, ray fungi, etc. Specifically, enzymes derived from Candida valida (Non-patent Document 1), Schizosaccharomyces pombe (Non-patent Document 2), Aspergillus niger (Non-patent Document 3), Aspergillus nidulans (Non-patent Document 3), Bacillus stearothermophilus (Non-patent Document 4), Cellulomonas sp. NT3060 (Non-patent Document 5), Escherichia coli (Non-patent Document 6), and Lactobacillus fermentum (Patent Document 1), etc., have been reported.

Among these, the entire amino acid sequences are known for glycerol dehydrogenase derived from Schizosaccharomyces pombe, Bacillus stearothermophilus, and Escherichia coli, which, however, are apparently different from the amino acid sequence of the enzyme of the present invention.

On the other hand, as for glycerol dehydrogenase derived from microorganisms of the genus Cellulomonas, one N-terminal amino acid and two C-terminal amino acids of the enzyme derived from the above-described Cellulomonas sp. NT3060 have been determined but its entire amino acid sequence has not been reported.

Furthermore, for a method for manufacturing (S)-3-chloro-1,2-propanediol, a method in which 1-chloro-3-hydroxyacetone is stereoselectively reduced using glycerol dehydrogenase derived from Cellulomonas sp. from SIGMA (Patent Document 2) or recombinant E. coli having a glycerol dehydrogenase gene derived from Serratia marcescens IFO12468 transferred therein (Patent Document 3) is known. However, in terms of industrial applications, there are great disadvantages such that in the former process the expensive commercial enzyme is required, and in the latter process the stability against the substrate, 1-chloro-3-hydroxyacetone, is not high so that a concentration of the substrate to be charged and a conversion rate from the substrate to the product are low.

[Patent Document 1] Japanese Patent Laid-Open No. 10-113170

[Patent Document 2] WO03/018523

[Patent Document 3] Japanese Patent Laid-Open No. 2003-61668

[Non-patent Document 1] J. Gen. Microbiol., 130, 3225 (1984)

[Non-patent Document 2] J. Gen. Microbiol., 131, 1581 (1985)

[Non-patent Document 3] J. Gen. Microbiol., 136, 1043 (1990)

[Non-patent Document 4] Biochim. Biochem. Acta, 994, 270 (1989)

[Non-patent Document 5] Agric. Biol. Chem., 48, 1603 (1982)

[Non-patent Document 6] J. Bacteriol., 140, 182 (1979)

### DISCLOSURE OF THE INVENTION

An object of the present invention is to provide a polypeptide that can stereoselectively reduce 1-chloro-3-hydroxyacetone to generate (S)-3-chloro-1,2-propanediol. An other object of the present invention is to efficiently produce the above polypeptide by utilizing recombinant DNA technology. Still another object of the present invention is to provide a practicable manufacturing method of (S)-3-chloro-1,2-propanediol by using the transformant.

We have found a novel polypeptide that can stereoselectively reduce 1-chloro-3-hydroxyacetone to generate (S)-3-chloro-1,2-propanediol. In addition, we have found that (S)-3-chloro-1,2-propanediol can be efficiently manufactured by using the polypeptide or a transformant that can produce the polypeptide. Thus, the present invention has been completed.

Particularly, the present invention is a polypeptide having physicochemical characteristics of (1) to (5) described below:

(1) action: to generate (S)-3-chloro-1,2-propanediol by stereoselectively reducing 1-chloro-3-hydroxyacetone using NADH as a coenzyme;

(2) molecular weight: about 340,000 by gel-filtration and about 43,000 by SDS polyacrylamide gel electrophoresis;

(3) optimum temperature: from 60 to 70° C.;

(4) optimum pH for reduction: 6.0; and

(5) optimum pH for oxidation: 9.0.

Furthermore, the present invention is the polypeptide (a) or (b) described below:

(a) a polypeptide comprising an amino acid sequence represented by SEQ ID NO: 1 in the Sequence Listing; or

(b) a polypeptide comprising an amino acid sequence in which one or more amino acids are substituted, inserted, deleted, and/or added in the amino acid sequence represented by SEQ ID NO: 1 in the Sequence Listing and having an enzyme activity to stereoselectively reduce 1-chloro-3-hydroxyacetone to generate (S)-3-chloro-1,2-propanediol.

Still furthermore, the present invention is a DNA encoding these polypeptides.

Moreover, the present invention is DNA that hybridizes with DNA comprising a nucleotide sequence complementary to a nucleotide sequence represented by SEQ ID NO: 2 in the Sequence Listing under stringent conditions and that encodes a polypeptide having an enzyme activity to stereoselectively reduce 1-chloro-3-hydroxyacetone to generate (S)-3-chloro-1,2-propanediol.

Still moreover, the present invention is DNA that has at least 60% sequence identity with the nucleotide sequence represented by SEQ ID NO: 2 in the Sequence Listing and that encodes a polypeptide having an enzyme activity to stereoselectively reduce 1-chloro-3-hydroxyacetone to generate (S)-3-chloro-1,2-propanediol.

In addition, the present invention is an expression vector comprising such a DNA and a transformant comprising the expression vector.

Also, the present invention is a manufacturing method of an optically active alcohol comprising the steps of reacting the above polypeptide or a culture of the above transformant with a compound having a carbonyl group and collecting the resulting optically active alcohol.

The present invention provides a novel glycerol dehydrogenase, DNA encoding the enzyme, and a transformant having the DNA. Furthermore, by using the enzyme or the transformant, optically active alcohols, in particular, (S)-3-chloro-1,2-propanediol, can be efficiently made from compounds having a carbonyl group therein.

### BRIEF DESCRIPTION OF THE DRAWINGS

FIG. 1 shows the nucleotide sequence of a gene encoding RCG and the putative amino acid sequence of RCG;

FIG. 2 shows construction of recombinant vectors, pTSCS and pTSCSG1; and

FIG. 3 shows stability of RCG against 1-chloro-3-hydroxyacetone and 3-chloro-1,2-propanediol.

### BEST MODE FOR CARRYING OUT THE INVENTION

The present invention will be explained in detail below.

The polypeptide of the present invention can be obtained from microorganisms having an activity to stereoselectively reduce 1-chloro-3-hydroxyacetone to generate (S)-3-chloro-1,2-propanediol. Microorganisms used as sources providing the polypeptide include, for example, Cellulomonas sp. strain KNK0102. The above strain KNK0102 is a microorganism isolated from the soil by the present inventors.

Mycological properties of Cellulomonas sp. strain KNK0102 are described below.

### A. Morphology

(1) A cell has a size of 0.5 to 0.7 μm by 1.5 to 2.0 μm and an irregular rod shape.

(2) A cell is motile, having one flagellum.

(3) No spore.

(4) Gram stain: positive.

(5) Colony morphology: circular, convex, entire, and yellow.

### B. Physiological Properties

(1) Hydrolysis of gelatin: positive.

(2) Hydrolysis of starch: positive.

(3) Reduction of nitrate: positive.

(4) Catalase activity: positive.

(5) Oxidase activity: negative.

(6) Urease activity: negative.

(7) DNAse activity: positive.

(8) Aminopeptidase activity: negative.

(9) Cellulose decomposition: negative.

(10) The Voges-Proskauer test: negative.

(11) The methyl red test: negative.

(12) The O—F test: fermentation.

(13) Decomposition of carbohydrates: in anaerobic conditions, acids are generated from fructose, galactose, glucose, glycogen, maltose, starch, sucrose, and xylose, but not from glycerin, lactose, mannitol, and mannose.

A microorganism producing the polypeptide of the present invention can be either a wild strain or a variant. Furthermore, a microorganism derived by a genetic technique such as cell fusion or gene manipulation can be used. A microorganism genetically manipulated to produce the polypeptide of the present invention can be obtained, for example, by the method comprising the steps of: isolating and/or purifying such an enzyme and determining a partial or entire amino acid sequence of the enzyme; determining a nucleotide sequence of DNA encoding the polypeptide based on an amino acid sequence thereof; obtaining DNA encoding the polypeptide based on the nucleotide sequence; obtaining a recombinant microorganism by transferring the DNA into a suitable host microorganism; and obtaining the enzyme of the present invention by culturing the recombinant microorganism.

The polypeptides of the present invention may include, for example, a polypeptide having physicochemical characteristics of (1) to (5) described below:

(1) action: to generate (S)-3-chloro-1,2-propanediol by stereoselectively reducing 1-chloro-3-hydroxyacetone using NADH as a coenzyme;

(2) molecular weight: about 340,000 by gel-filtration and about 43,000 by SDS polyacrylamide gel electrophoresis;

(3) optimum temperature: from 60 to 70° C.;

(4) optimum pH for reduction: 6.0; and

(5) optimum pH for oxidation: 9.0.

The reduction activity of the enzyme can be determined, for example, by adding 20 mM of the substrate, 0.167 mM of coenzyme NADH, and the enzyme solution to a 100 mM phosphate buffer (pH 6.5) followed by the measurement of a decrease in absorbance at 340 nm at 30° C.

The oxidation activity of the enzyme can be determined, for example, by adding 0.1 M of the substrate, 2 mM of coenzyme NAD⁺, and the enzyme solution to a 100 mM phosphate buffer (pH 8.0) followed by the measurement of an increase in absorbance at 340 nm at 30° C.

The molecular weight of the enzyme can be determined, for example, by calculating from relative elution times of standard proteins on gel-filtration using a Superdex 200HR10/30 column (from Pharmacia Biotech). Furthermore, the molecular weight of the subunit can be determined by calculating from relative mobilities of standard proteins on 20% SDS-polyacrylamide gel electrophoresis.

The optimum pH and temperature of the enzyme can be determined, for example, by measuring activities by varying reaction pH and temperature in the system described above to determine the reduction activity.

The polypeptide of the present invention has resistance to 1-chloro-3-hydroxyacetone and 3-chloro-1,2-propanediol. That is, in the reaction to generate (S)-3-chloro-1,2-propanediol by stereoselectively reducing 1-chloro-3-hydroxyacetone, the polypeptide of the present invention is not readily subject to substrate inhibition and/or product inhibition. Therefore, the polypeptide of the present invention is suitable in manufacturing (S)-3-chloro-1,2-propanediol.

Herein, the expression “having resistance to 1-chloro-3-hydroxyacetone” means that when 1-chloro-3-hydroxyacetone is added to a solution containing the polypeptide (the enzyme) of the present invention at the final concentration of 0.25% and the mixture is incubated at 30° C. for 20 hrs. followed by the determination of the activity to reduce 1-chloro-3-hydroxyacetone under the conditions described above, the resulting activity is 80% or more, preferably 85% or more, more preferably 90% or more of the activity determined without the pretreatment.

Likewise, the expression “having resistance to 3-chloro-1,2-propanediol” means that when the activity to reduce 1-chloro-3-hydroxyacetone is determined after incubation at 30° C. for 20 hrs. in the presence of 1% of 3-chloro-1,2-propanediol, the resulting activity is 80% or more, preferably 90% or more, more preferably 95% or more of the activity determined without the pretreatment.

Furthermore, the polypeptide of the present invention may be a polypeptide comprising an amino acid sequence represented by SEQ ID NO: 1 in the Sequence Listing; or a polypeptide comprising an amino acid sequence in which one or more amino acids are substituted, inserted, deleted, and/or added in the amino acid sequence represented by SEQ ID NO: 1 in the Sequence Listing and having an enzyme activity to stereoselectively reduce 1-chloro-3-hydroxyacetone to generate (S)-3-chloro-1,2-propanediol.

The polypeptide comprising an amino acid sequence in which one or more amino acids are substituted, inserted, deleted, and/or added in the amino acid sequence represented by SEQ ID NO: 1 in the Sequence Listing can be prepared according to the well-known method described in Current Protocols in Molecular Biology (John Wiley and Sons, Inc., 1989), etc. Such a polypeptide is included by the polypeptide in the present invention, as long as such a polypeptide has an enzyme activity to stereoselectively reduce 1-chloro-3-hydroxyacetone to generate (S)-3-chloro-1,2-propanediol.

The position where amino acids are substituted, inserted, deleted, and/or added in the amino acid sequence represented by SEQ ID NO: 1 in the Sequence Listing is not particularly limited, but preferably highly conserved regions are avoided. Herein, a highly conserved region refers to the region where amino acids are identical among a plurality of sequences, when the amino acid sequences of a plurality of dehydrogenases of different origins are optimally aligned and compared. A highly conserved region can be confirmed by comparing using a tool such as GENETYX the amino acid sequence represented by SEQ ID NO: 1 with the amino acid sequences of other glycerol dehydrogenases derived from Bacillus stearothermophilus and Escherichia coli described above.

An amino acid sequence modified by substitution, insertion, deletion, and/or addition may the one that includes only one type of modification (e.g., substitution), or two or more modifications (e.g., substitution and insertion).

Furthermore, in case of substitution, it is preferable that an amino acid present after substitution is a homologous amino acid to the original amino acid. Herein, amino acids belonging to each of the following groups are designated as homologous amino acids.

(Group 1) Leu, Ile, Val, Met, H is, Trp, Tyr, Phe

(Group 2) Glu, Gln, Asp, Asn

(Group 3) Ser, Thr, Cys, Gly, Ala, Pro

(Group 4) Lys, Arg

The number of amino acids subject to substitution, insertion, deletion, and/or addition is not particularly limited, as long as the polypeptide after modification has the enzyme activity to stereoselectively reduce 1-chloro-3-hydroxyacetone to generate (S)-3-chloro-1,2-propanediol. However, sequence identity with the amino acid sequence represented by SEQ ID NO: 1 in the Sequence Listing is preferably 80%. More preferable is 90% or more sequence identity, still more preferable is 95% or more sequence identity, and most preferable is 99% or more sequence identity. Sequence identity is expressed, when the amino acid sequence represented by SEQ ID NO: 1 in the Sequence Listing and a modified amino acid sequence are compared in a similar manner described above to confirm a highly conserved region, by a value obtained by dividing the number of locations where amino acids are identical between the both sequences by the total number of amino acids compared, which is further multiplied by 100.

Any polynucleotide encoding the above polypeptide can be used as a polynucleotide of the present invention. Examples include a polynucleotide comprising the nucleotide sequence represented by SEQ ID NO: 2 in the Sequence Listing, and a polynucleotide that hybridizes with a polynucleotide comprising the nucleotide sequence complementary to the nucleotide sequence represented by SEQ ID NO: 2 in the Sequence Listing under stringent conditions.

Herein, the expression “a polynucleotide that hybridizes with a polynucleotide comprising the nucleotide sequence complementary to the nucleotide sequence represented by SEQ ID NO: 2 in the Sequence Listing under stringent conditions” means a polynucleotide obtained under stringent conditions by using colony hybridization, plaque hybridization, Southern hybridization, or the like with a polynucleotide comprising the nucleotide sequence complementary to the nucleotide sequence represented by SEQ ID NO: 2 in the Sequence Listing working as a probe.

Hybridization can be performed according to the method described in Molecular Cloning, A laboratory manual, second edition (Cold Spring Harbor Laboratory Press, 1989), etc. Herein, the expression “a polynucleotide that hybridizes under stringent conditions” refers to, for example, a polynucleotide that can be obtained, after hybridization is performed in the presence of 0.7 to 1.0 M of NaCl at 65° C. by using a filter on which a polynucleotide derived from a colony or a plaque is immobilized, by washing the filter with 2×SSC solution (the SSC solution contains 150 mM sodium chloride and 15 mM sodium citrate) under the conditions of 65° C. A nucleotide can be obtained by washing preferably at 65° C. with 0.5×SSC solution, more preferably at 65° C. with 0.2×SSC solution, and still more preferably at 65° C. with 0.1×SSC solution.

Polynucleotides that can hybridize under the above-described conditions include polypeptides whose sequence identity with the polynucleotide indicated by SEQ ID NO: 2 is 60% or more, preferably 80% or more, more preferably 90% or more, still more preferably 95% or more, and most preferably 99% or more. However, as long as a polypeptide encoded has the activity to stereoselectively reduce 1-chloro-3-hydroxyacetone to generate (S)-3-chloro-1,2-propanediol, such a polynucleotide is included by the polynucleotide of the present invention.

Herein, “sequence identity (%)” is expressed by a value obtained by compared optimally aligning two polynucleotides to be, dividing the number of locations where nucleic acid bases (e.g., A, T, C, G, U, or I) are identical between the both sequences by the total number of bases compared, and multiplying the value thus obtained by 100.

Sequence identity, for example, can be calculated by the following analytical tools: GCG Wisconsin Package (Program Manual for The Wisconsin Package, Version8, September 1994, Genetics Computer Group, 575 Science Drive Medison, Wis., USA 53711; Rice, P. (1996) Program Manual for EGCG Package, Peter Rice, The Sanger Centre, Hinxton Hall, Cambridge, CB10 1RQ, England); and the ExPASy World Wide Web molecular biology server (Geneva University Hospital and University of Geneva, Geneva, Switzerland).

An example of the method for obtaining from Cellulomonas sp. strain KNK0102 the polypeptide (the enzyme) of the present invention is described below, but the present invention is not limited by this.

First, a microorganism having an enzyme that stereoselectively reduces 1-chloro-3-hydroxyacetone to generate (S)-3-chloro-1,2-propanediol is cultured in a suitable medium. For culturing, as long as the microorganism grows, a common liquid nutrient medium containing a carbon source, a nitrogen source, inorganic salts, organic nutrients, etc., can be used. Culture can be performed, for example, at temperature of 25 to 37° C., at pH of 4 to 8, by shaking or aerating.

After microbial cells are separated from the culture solution by centrifugation, the microbial cells are suspended in a suitable buffer, the microbial cells are crushed or dissolved either physically using glass beads, etc., or biochemically with an enzyme, etc., and solids in the solution are removed further by centrifugation, and thereby a crude enzyme solution of the enzyme can be obtained. In addition, the crude enzyme solution can be further purified by a method that those skilled in the art usually employ, for example, by one of ammonium sulfate precipitation, dialysis, chromatography, and the like, or a combination thereof. As for chromatography, one of hydrophobic chromatography, ion-exchange chromatography, and gel-filtration chromatography, or a combination thereof can be also used.

An example of the method for obtaining DNA encoding the enzyme of the present invention is described below, but the present invention is not limited by this.

Primers for PCR (Polymerase Chain Reaction) are synthesized based on the conserved sequence of amino acids commonly present among various glycerol dehydrogenases. Then, from a microorganism from which the DNA is derived, chromosomal DNA of the microorganism is prepared by a common method for isolating DNA, e.g., the Hereford method (Cell, 18, 1261 (1979)). PCR is performed using the chromosomal DNA as a template and the above PCR primers, and a part of the DNA encoding the polypeptide (core sequence) is amplified to determine a nucleotide sequence. The nucleotide sequence can be determined by the dideoxy chain termination method, etc. For example, an ABI 373A DNA Sequencer (from Applied Biosystems), etc., can be used.

In order to elucidate the nucleotide sequence in the flanking regions of the core sequence, chromosomal DNA of the microorganism is digested by a restriction enzyme whose recognition sequence is not present in the core sequence. The resulting DNA fragment is self ligated by T4 ligase to prepare a template DNA for inverse PCR (Inverse PCR: Nucleic Acids Res., 16, 8186 (1988)). Then, based on the core sequence, primers that become starting points for DNA synthesis extending outward from the core sequence are synthesized, and the flanking regions of the core sequence are amplified by inverse PCR. By elucidating the nucleotide sequence of the resulting DNA, a DNA sequence of the entire encoding region of glycerol dehydrogenase of interest can be known.

As a vector DNA used for transferring DNA of the enzyme of the present invention into a host microorganism, so that the DNA is expressed inside the host microorganism with the DNA transferred therein, any vector DNA able to express the enzyme gene inside a suitable host microorganism can be employed. Such a vector DNA includes, for example, plasmid vectors, phage vectors, cosmid vectors, etc. In addition, shuttle vectors, wherein the gene can be exchanged with another host strain, can be used.

Such a vector contains regulatory elements for operably linked promoters (lacUV5 promoter, trp promoter, trc promoter, tac promoter, lpp promoter, tufB promoter, recA promoter, pL promoter, etc.), and can be suitably used as an expression vector that contains an expression unit operably linked with the DNA of the present invention. For example, pUCNT (WO94/03613), etc., can be preferably used.

The term used herein a “regulatory element” refers to a nucleotide sequence having functional promoters and any related transcription elements (for example, an enhancer, CCAAT box, TATA box, SPI site).

The term used herein “operably linked” refers to that, in order that a gene is expressed, DNA is linked with various regulatory elements that regulate expression thereof, such as promoters and enhancers, in a manner that the regulatory elements can act thereon inside a host microorganism. It is well know by those skilled in the art that the type and kind of regulatory element depend on a host.

Host cells into which a vector having the DNA of the present invention is transferred include bacteria, yeasts, filamentous fungi, plant cells, animal cells, and the like, and E. coli is particularly preferable. The DNA of the present invention can be transferred into host cells by the conventional method. When E. coli is used as a host cell, the DNA of the present invention can be transferred, for example, by the calcium chloride method.

When, by using the enzyme of the present invention or a recombinant capable of producing the enzyme, a compound having a carbonyl group is stereoselectively reduced to generate an optically active alcohol, NADH is required as coenzyme. By adding to the reaction system a necessary amount of NADH only, reduction may proceed. However, on conducting the reaction, the amount of expensive coenzyme used for the reaction can be significantly reduced, by adding enzyme having an ability to convert the coenzyme that has been oxidized (NAD⁺) into the reduced form (NADH) (herein after referred to as the regeneration ability of coenzyme) together with the substrate thereof, that is, by combining the regeneration system of coenzyme with the enzyme of the present invention. As an enzyme having the regeneration ability of coenzyme, hydrogenase, formate dehydrogenase, alcohol dehydrogenase, glucose-6-phosphate dehydrogenase, glucose dehydrogenase, etc., can be used. Preferably, glucose dehydrogenase and formate dehydrogenase are used. Such a reaction may also be conducted by adding the regeneration system of coenzyme in the asymmetric reduction reaction system, but when a transformant transformed by both the DNA encoding the enzyme of the present invention and the DNA encoding glucose dehydrogenase is used as a catalyst, the reaction can be efficiently conducted without separately preparing the enzyme having the regeneration ability of coenzyme and adding it to the reaction system. Such a transformant can be obtained by incorporating into a single vector the DNA encoding the enzyme of the present invention and the DNA encoding the glucose dehydrogenase, which is then transferred into a host cell, or by incorporating each of these two kinds of DNA into each of two different incompatible vectors separately, which are then transferred into a single host cell.

The production of an optically active alcohol from a compound having a carbonyl group by using the enzyme of the present invention or a culture of the transformant of the present invention is conducted as described below. However, the present invention is not limited by the method below.

First, to a suitable solvent, a compound having a carbonyl group as a substrate, a coenzyme such as NAD⁺, and a culture of the transformant are added, and while pH is being adjusted, the reaction is allowed to proceed by stirring. This reaction is conducted at temperature from 10 to 70° C., and pH of the reaction solution is kept at 4 to 10 during the reaction. The reaction can be conducted in a batch or continuously. In the case that the reaction is conducted in a batch, the reaction substrate can be added at a charging concentration of from 0.1 to 70 w/v %.

The term used herein a “culture” refers to cells of a microorganism, a culture solution containing microbial cells, or treated microbial cells. Treated microbial cells include, for example, dried microbial cells obtained by dehydration with acetone or diphosphorus pentaoxide or by drying utilizing a desiccator or a fan; a surfactant treated cells; a lytic enzyme treated cells; immobilized microbial cells; cell-free extract preparation, in which microbial cells are crushed; or the like. Furthermore, an enzyme that catalyzes asymmetric reduction can be purified from a culture to provide it for use.

Moreover, for conducting the reaction, when a transformant that produces both the enzyme of the present invention and glucose dehydrogenase is used, the amount of a coenzyme used for the reaction can be significantly reduced by further adding glucose to the reaction system.

The method for collecting optically active alcohols obtained by any of the methods described above is not particularly limited. The alcohols can be, either directly from the reaction solution or after the microbial cells are separated, extracted with a solvent such as ethyl acetate, toluene, t-butyl methyl ether, and hexane, and dehydrated followed by purification by evaporation, crystallization, column chromatography with silica gel, or the like. Thus, highly purified, optically active alcohols can be readily obtained.

### EXAMPLES

Below, the present invention is explained further in detail with examples, but the present invention should not be limited by these examples. In addition, in the description below, “%” means “% by weight” unless otherwise specified.

### Example 1

### Cloning of a Novel Glycerol Dehydrogenase

Based on the conserved sequence of amino acids commonly present in glycerol dehydrogenase, a DNA sequence was expected from the amino acid sequence and Primer 1 (5′-ACNGAYGARGGNGMNTTYGA-3′: SEQ ID NO: 3) and Primer 2 (5′-GGCATNTKRTGDATNGTYTC-3′: SEQ ID NO: 4) are synthesized. A buffer for ExTaq (50 μl) containing: two primers (Primer 1 and Primer 2), each 200 μmol; 1.2 μg of chromosomal DNA from Cellulomonas sp. strain KNK0102; dNTPs, each 10 nmol; and 2.5 U of ExTaq (from Takara Shuzo) was prepared, 30 cycles of thermal denaturation (97° C., 0.5 min.), annealing (45° C., 1 min.), and extension (72° C., 1 min.) were conducted followed by cooling to 4° C., and then amplified DNA was confirmed by agarose gel electrophoresis.

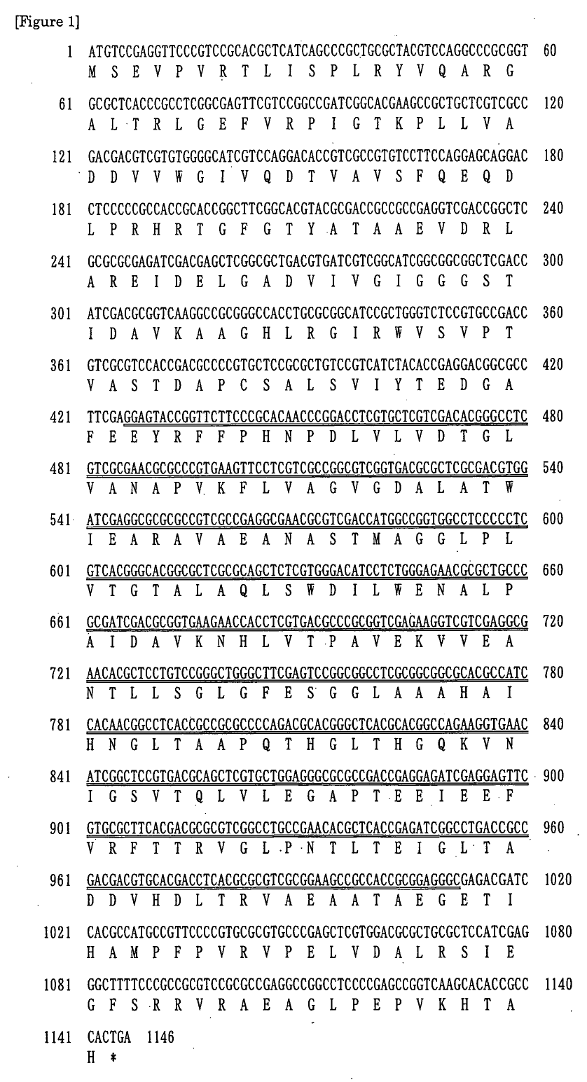

Chromosomal DNA from Cellulomonas sp. strain KNK0102 used for this reaction was prepared according to the preparation method in small quantities of bacterial genome DNA described in Bunshiseibutsugaku Jikken Purotokoru 1 (in Japanese) (Molecular Biology Experiment Protocols 1) (Maruzen) p. 36. The amplified DNA was subcloned into a pT7Blue Vector (from Novagen), and the nucleotide sequence thereof was determined. The result showed that the amplified DNA was made of 586 bases excluding the primer sequences. The sequence thereof is a portion of the DNA sequence underlined with double lines in the DNA sequence shown in FIG. 1. Hereinafter, this sequence is referred to as the “core sequence.”

Based on the nucleotide sequence near the 5′-side of the core sequence, the complimentary sequence thereof, Primer 3 (5′-TTCTCCCAGAGGATGTCCCACGAG-3′: SEQ ID NO: 5), was made, and also based on the nucleotide sequence near the 3′-side, Primer 4 (5′-AGATCGAGGAGTTCGTGCGCTTCA-3′: SEQ ID NO: 6) was made. For a template for inverse PCR, chromosomal DNA of Cellulomonas sp. strain KNK0102 was first digested by the restriction enzyme XmaI, and the digest was self ligated using T4DNA ligase. A buffer for ExTaq (50 μl) containing: 385 ng of this self ligated product; two primers (Primer 3 and Primer 4), each 50 pmol; dNTPs, each 10 nmol; and 2.5 U of ExTaq (from Takara Shuzo) was prepared, 35 cycles of thermal denaturation (97° C., 0.5 min.), annealing (70° C., 1 min.), and extension (72° C., 5 min.) were conducted followed by cooling to 4° C., and then amplified DNA was confirmed by agarose gel electrophoresis.

The amplified DNA was subcloned into a pT7Blue Vector (from Novagen), and the nucleotide sequence thereof was determined. From this result and the result of the core sequence, the entire nucleotide sequence of the gene encoding a glycerol dehydrogenase derived from Cellulomonas sp. strain KNK0102 (herein after this enzyme is referred to as RCG) was determined. The entire nucleotide sequence and the putative amino acid sequence encoded by the gene are shown together in FIG. 1.

### Example 2

### Preparation of a Recombinant Vector Comprising the RCG Gene

In order that RCG is expressed in E. coli, a recombinant vector to be used for transformation was prepared. Double-stranded DNA, in which a NdeI site was added to the initiation codon region of the RCG gene, and a new termination codon and an EcoRI site were added just after the termination codon, was obtained by the method described below.

Based on the nucleotide sequence determined in Example 1, Primer 5 (5′-GATCATATGTCCGAGGTTCCCGTCCGC-3′: SEQ ID NO: 7) in which a NdeI site was added to the initiation codon region of the RCG gene, and Primer 6 (5′-CTAGAATTCTTATCAGTGGGCGGTGTGCTTGAC-3′: SEQ ID NO: 8) in which a new termination codon (TAA) and an EcoRI site were added just after the termination codon of the RCG gene were synthesized. A buffer for ExTaq (50 μl) containing: two primers (Primer 5 and Primer 6), each 50 μmol; 950 ng of chromosomal DNA of Cellulomonas sp. strain KNK0102; dNTPs, each 10 nmol; and 2.5 U of ExTaq (from Takara Shuzo) was prepared, 35 cycles of thermal denaturation (97° C., 0.5 min.), annealing (65° C., 1 min.), and extension (72° C., 1 min.) were conducted followed by cooling to 4° C., and then amplified DNA was confirmed by agarose gel electrophoresis. This amplified fragment was digested by NdeI and EcoRI, which was inserted at NdeI and EcoRI sites downstream of the lac promoter of a plasmid pUCNT (WO94/03613) to obtain the recombinant vector pNTCS.

### Example 3

### Addition of a Shine-Dalgarno Sequence in the Upstream of the RCG Gene

In order that the RCG gene is expressed in E. coli at a high level, a plasmid in which a Shine-Dalgarno sequence (9 bases) of E. coli is additionally added upstream of the initiation codon of the same gene in the plasmid pNTCS prepared in Example 2 was obtained by the method described below.

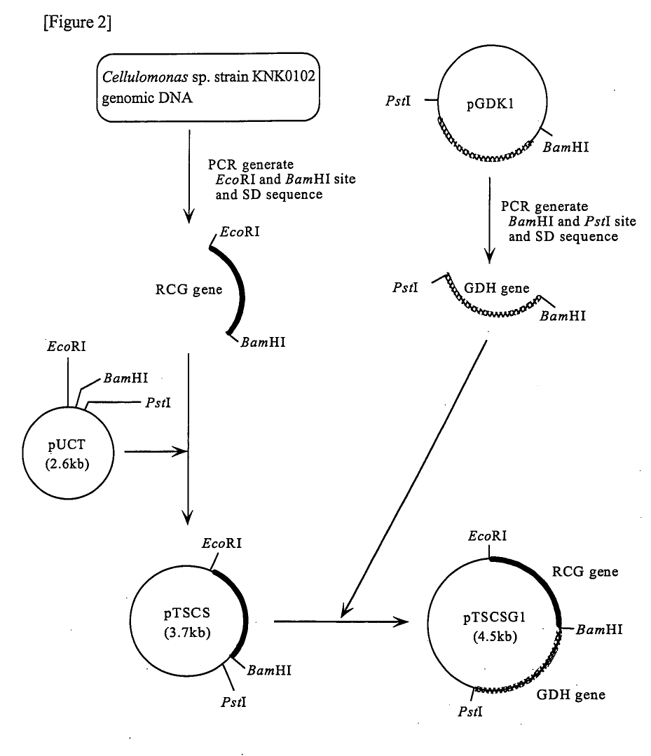

First, G in the NdeI site of the E. coli expression vector pUCNT used in Example 2 was converted into T by PCR to construct the plasmid pUCT. Next, based on the nucleotide sequence determined in Example 1, Primer 7 (5′-GCCGAATTCTAAGGAGGTTAACAATGTCCGAGGTTCCCGTCCG-3′: SEQ ID NO: 9) in which at 5 bases upstream from the initiation codon of the RCG gene, a Shine-Dalgarno sequence (9 bases) of E. coli, and also just before it, an EcoRI site were added; and Primer 8 (5′-GCGGGATCCTTATCAGTGGGCGGTGTGCTTGA-3′: SEQ ID NO: 10) in which just after the termination codon of the RCG gene, a new termination codon (TAA) and a BamHI site were added were synthesized. A buffer for ExTaq (50 μl) containing: two primers (Primer 7 and Primer 8), each 50 μmol; 950 ng of chromosomal DNA of Cellulomonas sp. strain KNK0102; dNTPs, each 10 nmol; and 2.5 U of ExTaq (from Takara Shuzo) was prepared, 35 cycles of thermal denaturation (97° C., 0.5 min.), annealing (65° C., 1 min.), and extension (72° C., 1 min.) were conducted followed by cooling to 4° C., and then amplified DNA was confirmed by agarose gel electrophoresis. This amplified fragment was digested by EcoRI and BamHI, which was inserted at EcoRI and BamHI sites in the downstream of the lac promoter of a plasmid pUCT to obtain the recombinant vector pTSCS. The preparation method and the structure of pTSCS are shown in FIG. 2.

### Example 4

### Preparation of a Recombinant Vector Simultaneously Comprising Both of the RCG Gene and the Glucose Dehydrogenase Gene

Double-stranded DNA, in which at 5 bases upstream from the initiation codon of the gene for a glucose dehydrogenase (herein after referred to as GDH) derived from Bacillus meqaterium strain IAM1030, a Shine-Dalgarno sequence (9 bases) of E. coli, and also just before it, a BamHI site, and just after the termination codon, a PstI site were added, was obtained by the method described below.

Based on the nucleotide sequence information of the GDH gene, Primer 9 (5′-GCCGGATCCTAAGGAGGTTAACAATGTATAAADATTTAGAAGG-3′: SEQ ID NO: 11) in which at 5 bases upstream from the initiation codon of the GDH structural gene, a Shine-Dalgarno sequence (9 bases) of E. coli, and also just before it, a BamHI site were added, and Primer 10 (5′-GCGCTGCAGTTATCCGCGTCCTGCTTGGA-3′: SEQ ID NO: 12) in which just after the termination codon, a PstI site was added were synthesized by the conventional method. By using these two primers, a plasmid pGDK1 (Eur. J. Biochem., 186, 389 (1989)) being a template, double-stranded DNA was synthesized by PCR. The resulting DNA fragment was digested by BamHI and PstI, which was inserted at the BamHI-PstI sites of pTSCS constructed in Example 3 to obtain the recombinant vector pTSCSG1. The preparation method and the structure of pTSCSG1 are shown in FIG. 2.

### Example 5

### Preparation of Recombinant E. coli

By using the recombinant vector pTSCS obtained in Example 3 and the recombinant vector pTSCSG1 obtained in Example 4, E. coli HB101 (from Takara Shuzo) was transformed to obtain recombinant E. coli HB101 (pTSCS) and recombinant E. coli HB101 (pTSCSG1), respectively. The resulting recombinant, E. coli HB101 (pTSCS), was deposited at International Patent Organism Depositary of National Institute of Advanced Industrial Science and Technology on May 12, 2004, Deposit number being FERM BP-10024.

### Example 6

### Expression of RCG in Recombinant E. coli

Each of the recombinant E. coli HB101 (pTSCS) and HB101 (pTSCSG1) obtained in Example 5, and E. coli HB101 (pUCT), a transformant containing a simple vector plasmid, was cultured in a 2×YT medium (1.6% of Bacto trypton, 1.0% Bacto yeast extract, 0.5% NaCl; pH 7.0) containing 100 μg/ml of ampicillin. Microorganisms were collected, and then suspended in a 100 mM phosphate buffer (pH 6.5), which was disrupted by ultrasonication using a UH-50 ultrasonic homogenizer (from SMT) to obtain a cell-free extract. The RCG activity of this cell-free extract was determined by adding 20 mM of the substrate 1-chloro-3-hydroxyacetone, 0.167 mM of coenzyme NADH, and an enzyme solution to a 100 mM phosphate buffer (pH 6.5) followed by the measurement of a decrease in absorbance at the wavelength of 340 nm at 30° C. The enzyme activity to oxidize 1 mmol of NADH to NAD⁺ for a minute under these reaction conditions was defined as 1 unit.

Moreover, the GDH activity was determined by adding 0.1 M of the substrate glucose, 2 mM of coenzyme NADP⁺, and an enzyme to a 1 M Tris-HCl buffer (pH 8.0) followed by the measurement of an increase in absorbance at the wavelength of 340 mM at 25° C. The enzyme activity to reduce 1 μmol of NADP⁺ to NADPH for a minute under these reaction conditions was defined as 1 unit.

As shown in Table 1, with E. coli HB101 (pTSCS) and with HB101 (pTSCSG1), an increase in RCG activity was observed as compared with E. coli HB101 (pUCT), a transformant containing a simple vector plasmid.

TABLE 1

| | Relative activity of the | GDH relative |
|---|---|---|
| Strain | CPD oxidation (U/mg) | activity (U/mg) |
| HB101 (pUCT) | <0.1 | <0.01 |
| HB101 (pTSCS) | 23.5 | <0.01 |
| HB101 (pTSCSG1) | 16.3 | 90.5 |

### Example 7

### Purification of RCG from Recombinant E. coli HB101 (pTSCS)

Into shaking flasks, 60 ml of a liquid medium (pH 7.0) containing 1.6% of Bacto trypton, 1.0% of Bacto yeast extract, and 0.5% of NaCl was divide followed by steam pasteurization at 120° C. for 20 min. To this liquid medium, ampicillin was added at the final concentration of 100 μg/ml, and recombinant E. coli HB101 (pTSCS) was inoculated followed by shake culture at 37° C. for 30 hrs. From 60 ml of the resulting culture solution, microbial cells were collected by centrifugation, which were washed with 120 ml of a 10 mM phosphate buffer (pH 7.0), and suspended in 60 ml of a 10 mM phosphate buffer (pH 7.0). Then, the microbial cells were disrupted by ultrasonication using a SONIFIRE 250 (from BRANSON), and residue of the microbial cells were removed by centrifugation to obtain 60 ml of a cell-free extract. This was provided to a TOYOPEARL Super-Q 650S column (from Tosoh) equilibrated in advance with a 10 mM phosphate buffer (pH 7.0) and the enzyme was adsorbed thereon, which was then eluted with linear gradient of NaCl (from 0 mM to 400 mM) to obtain a fraction having an activity to reduce 1-chloro-3-hydroxyacetone. By this purification process, a purified enzyme preparation, which was singular in terms of electrophoresis, was obtained. The molecular weight of the band on SDS electrophoresis was about 43,000.

### Example 8

### Determination of Characteristics of RCG

The enzyme chemical characteristics of the resulting RCG were studied.

(1) The Action

The enzyme acted on 1-chloro-3-hydroxyacetone using NADH as a coenzyme to reduce it into (S)-3-chloro-1,2-propanediol having optical purity of 99.3% e.e.

(2) The Molecular Weight

By using a 50 mM phosphate buffer (pH 7.0) containing 150 mM NaCl as an eluent, the purified enzyme was analyzed by gel-filtration chromatography using Superdex 200 HR 10/30 (from Pharmacia Biotech). As a result, by calculating from relative retention times of standard proteins, the molecular weight of the enzyme was about 340,000.

(3) The Optimum Temperature

In order to study the optimum temperature of RCG, the activity to oxidize glycerol was determined at 10 to 70° C. The activity was determined by adding 0.1 M of the substrate glycerol, 2 mM of coenzyme NAD⁺, and the enzyme solution to a 100 mM phosphate buffer (pH 8.0) to react at 10 to 70° C. for 3 min. followed by the measurement of an increase in absorbance at 340 mM. The results showed that the optimum temperature was 60 to 70° C.

(4) The Optimum pH for Reduction

In order to study the optimum pH of RCG for reduction, the activity to reduce dihydroxyacetone at pH 4 to pH 9 was determined. The enzyme activity was determined by adding 10 mM of the substrate dihydroxyacetone, 0.167 mM of coenzyme NADH, 50 mM of KCl, 50 mM of NH₄Cl, and the enzyme solution to a buffer to react at 30° C. for 3 min. followed by the measurement of a decrease in absorbance at 340 mM. As buffers, 100 mM acetic acid buffers (pH4.0 to pH 6.0), 100 mM phosphate buffers (pH 6.0 to pH 8.0), and 100 mM Tris-HCl buffers (pH 8.0 to 9.0) were used to measure the activity at pH 4 to pH 9. The results showed that the optimum pH for reduction was pH 6.0.

### (5) The Optimum pH for Oxidation

In order to study the optimum pH of RCG for oxidation, the activity to oxidize glycerol at pH6 to pH 11 was determined. The enzyme activity was determined by adding 0.1 M of the substrate glycerol, 2 mM of coenzyme NAD⁺, 50 mM of KCl, 50 mM of NH₄Cl, and the enzyme solution to a buffer to react at 30° C. for 3 min. followed by the measurement of an increase in absorbance at 340 mM. As buffers, 100 mM phosphate buffers (pH 6.0 to pH 8.0), 100 mM Tris-HCl buffers (pH 8.0 to 9.0), and 100 mM carbonic acid buffers (pH 9.0 to pH 11.0) were used to measure the activity at pH 6 to pH 11. The results showed that the optimum pH for oxidation was pH 9.0.

### Example 9

### Substrate Specificity of RCG

Oxidation activity was determined by adding 2 mM of coenzyme NAD⁺, 10 mM of a compound shown in Table 2, and the enzyme solution to a 100 mM phosphate buffer (pH 8.0) to react at 30° C. for 3 min. followed by the measurement of an increase in absorbance at the wavelength of 340 mM. In Table 2, relative activities are summarized, wherein the activity for glycerol is 100.

TABLE 2

| | Relative activity |
|---|---|
| Substrate (10 mM) | (%) |
| glycerol | 100 |
| ethylene glycol | 8 |
| 1,2-propanediol | 142 |
| 1,3-propanediol | 1 |
| 3-methoxy-1,2-propanediol | 39 |
| 3-mercapto-1,2-propanediol | 129 |
| 3-methylthio-1,2-propanediol | 120 |
| 3-chloro-1,2-propanediol | 63 |
| 3-bromo-1,2-propanediol | 58 |
| 3-phenoxy-1,2-propanediol | 9 |
| 1,2-butanediol | 129 |
| 1,3-butanediol | 3 |
| 1,4-butanediol | 1 |
| 2,3-butanediol | 64 |
| 1,2-pentanediol | 153 |
| 1,4-pentanediol | 0 |
| 1,2-hexanediol | 26 |
| methanol | 0 |
| ethanol | 0 |
| propanol | 0 |
| isopropanol | 0 |
| 1-amino-2-propanol | 12 |
| 1,3-dichloro-2-propanol | 0 |
| butanol | 0 |
| 2-butanol | 0 |
| 1,2,4-butanetriol | 21 |

### Example 10

### Synthesis of (S)-3-chloro-1,2-propanediol from 1-chloro-3-hydroxyacetone by Using Recombinant E. coli with the RCG Gene Transferred Therein

To 1 ml of a cell-free extract of recombinant E. coli HB101 (pTSCS) obtained in Example 6, 100 units of glucose dehydrogenase (from Amano Enzyme), 10 mg of 1-chloro-3-hydroxyacetone, 1 mg of NAD⁺, and 20 mg of glucose were added to shake at 30° C. for 5 hrs. After the reaction was completed, the reaction solution was saturated with ammonium sulfate, and ethyl acetate was added to perform extraction. The resulting (S)-3-chloro-1,2-propanediol was analyzed by capillary gas chromatography.

### [Conditions for Analysis]

Column: HP-5; 30 m by 0.32 mm I. D. (from Agilent Technologies);

Detection: FID;

Initial column temperature: 50° C.; final column temperature: 200° C.; temperature raising rate: 6° C./min.;

Charging temperature: 150° C.;

Detection temperature: 300° C.;

Carrier gas: helium (70 kPa);

Split ratio: 100/1; and

Detection time: 1-chloro-3-hydroxyacetone: 8.2 min.; 3-chloro-1,2-propanediol: 10.2 min.

Furthermore, optical purity of the resulting 3-chloro-1,2-propanediol was determined by high performance liquid chromatography after tosylation.

### [Conditions for Analysis]

Column: Chiralpak AD (from Daicel Chemical Industries);

Eluent: hexane/isopropanol=9/1;

Flow rate: 0.8 ml/min.;

Detection: 235 nm;

Column temperature: 30° C.;

Elution time: S-stereoisomer: 29 min.; R-stereoisomer: 34 min.; and

Optical purity (% ee)=(A−B)/(A+B)×100

(A and B refer to amounts of corresponding enantiomers, and A is greater than B).

The results showed that the amount of (S)-3-chloro-1,2-propanediol generated was 7.6 mg; and optical purity was 99.4% ee.

### Example 11

### Synthesis of (S)-3-chloro-1,2-propanediol from 1-chloro-3-hydroxyacetone by Using Recombinant E. coli with RCG and a Glucose Dehydrogenase Simultaneously Expressed Therein

To 50 ml of a cell-free extract of recombinant E. coli HB101 (pTSCSG1) obtained in Example 6, 10 g of glucose, 5 mg of NAD, and 5 g of 1-chloro-3-hydroxyacetone were added. This reaction solution was stirred at 30° C. for 24 hrs, while pH was being adjusted to 6.5 by dropping 5 M NaOH. After the reaction was completed, the reaction solution was saturated with ammonium sulfate, and ethyl acetate was added to perform extraction followed by the analysis similarly to Example 10. The results showed that the amount of (S)-3-chloro-1,2-propanediol generated was 4.3 g; and optical purity was 99.3% ee.

### Example 12

### Evaluation of the Stability of RCG to 1-chloro-3-hydroxyacetone and to 3-chloro-1,2-propanediol

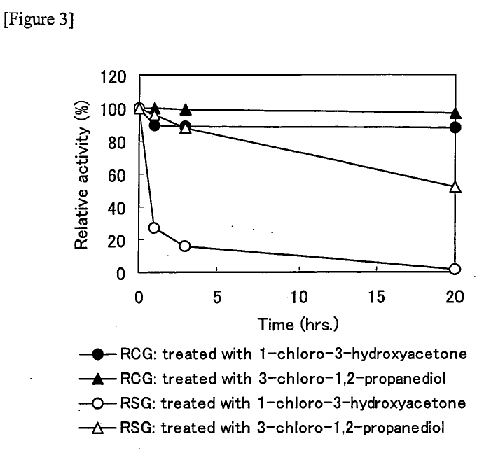

In order to study the stability of RCG to 1-chloro-3-hydroxyacetone and to 3-chloro-1,2-propanediol, 1-chloro-3-hydroxyacetone was added at the final concentration of 0.25% to the enzyme solution and incubated at 30° C. followed by the measurement of the activity to reduce 1-chloro-3-hydroxyacetone by using the above enzyme solution. Also, 3-chloro-1,2-propanediol was added at the final concentration of 1% to the enzyme solution and incubated at 30° C. followed by the measurement of the activity to reduce 1-chloro-3-hydroxyacetone. As shown in FIG. 3, the results showed that as compared with a glycerol dehydrogenase RSG (WO03/018523) derived from Serratia marcescens, which is known to stereoselectively reduce 1-chloro-3-hydroxyacetone into an S-stereoisomer, RCG was very stable to 1-chloro-3-hydroxyacetone and to 3-chloro-1,2-propanediol.

| | | |
|---|---|---|
| 0-1 | Form PCT/RO/134 (SAFE) | |
| 0-1-1 | The indications relating to the | JPO-PAS 0324 |
| | deposited microorganisms or | |
| | other biological material (PCT | |
| | Rule 13bis) were made by the | |
| | following. | |
| 0-2 | International application No. | PCT/JP2005/010960 |
| 0-3 | Applicant's or agent's file | B040280WO01- |
| | reference | |
| 1 | The indications made below | |
| | relate to the microorganism or | |
| | biological material referred | |
| | to in the detailed description | |
| | of the invention. | |
| 1-1 | Paragraph Number | 0073 |
| 1-3 | Indications of deposit | |
| 1-3-1 | Name of depositary | International Patent |
| | institution | Organism Depositary |
| | | (IPOD), National |
| | | Institute of Advanced |
| | | Industrial Science and |
| | | Technology |
| 1-3-2 | Address of depositary | Tsukuba Central 6, |
| | institution | 1-1-1 Higashi, |
| | | Tsukuba, Ibaraki |
| | | 305-8566, Japan |
| 1-3-3 | Date of deposit | Dec. 05, 2004 |
| | | (12.05.2004) |
| 1-3-4 | Accession Number | IPOD FERM BP-10024 |
| 1-5 | Designated States for which | all designated States |
| | indications are made | |

For receiving Office use only

| | | |
|---|---|---|
| 0-4 | This sheet was received with | yes |
| | the international application | |
| | (Yes/No) | |
| 0-4-1 | Authorized officer | Misako Akutsu |

For International Bureau use only

| | |
|---|---|
| 0-5 | This sheet was received by the |
| | International Bureau on: |
| 0-5-1 | Authorized officer |

1. A polypeptide having physicochemical characteristics of (1) to (5) described below:
(1) action: act on 1-chloro-3-hydroxyacetone using NADH as a coenzyme to generate (S)-3-chloro-1,2-propanediol;
(2) molecular weight: about 340,000 by gel-filtration and about 43,000 by SDS polyacrylamide gel electrophoresis;
(3) optimum temperature: from 60 to 70° C.;
(4) optimum pH for reduction: 6.0; and
(5) optimum pH for oxidation: 9.0.

2. The polypeptide set forth in claim 1, which is resistant to 1-chloro-3-hydroxyacetone.

3. The polypeptide set forth in claim 1, wherein the polypeptide is derived from Cellulomonas sp. strain KNK0102.

4. A polypeptide (a) or (b) described below:
(a) a polypeptide comprising an amino acid sequence represented by SEQ ID NO: 1 in the Sequence Listing; or
(b) a polypeptide comprising an amino acid sequence in which one or more amino acids are substituted, inserted, deleted, and/or added in the amino acid sequence represented by SEQ ID NO: 1 in the Sequence Listing, and having an enzyme activity to stereoselectively reduce 1-chloro-3-hydroxyacetone to generate (S)-3-chloro-1,2-propanediol.

5. A DNA encoding the polypeptide set forth in claim 1.

6. A DNA (a) or (b) described below:
(a) a DNA comprising a nucleotide sequence represented by SEQ ID NO: 2 in the Sequence Listing; or
(b) a DNA that hybridizes with a DNA comprising a nucleotide sequence complementary to the nucleotide sequence represented by SEQ ID NO: 2 in the Sequence Listing under stringent conditions and that encodes a polypeptide having an enzyme activity to stereoselectively reduce 1-chloro-3-hydroxyacetone to generate (S)-3-chloro-1,2-propanediol.

7. An expression vector containing the DNA set forth in claim 5.

8. The expression vector set forth in claim 7, wherein the expression vector is the plasmid pTSCS shown in FIG. 2.

9. A transformant obtained by transforming a host cell by using the expression vector set forth in claim 7.

10. The transformant set forth in claim 9, wherein said host cell is Escherichia coli.

11. The transformant set forth in claim 10, wherein the Escherichia coli is Escherichia coli HB101 (pTSCS)(FERM BP-10024).

12. A manufacturing method of an optically active alcohol characterized by reacting the polypeptide set forth in claim 1 with a compound having a carbonyl group.

13. The manufacturing method set forth in claim 12, wherein (S)-3-chloro-1,2-propanediol is manufactured from 1-chloro-3-hydroxyacetone.

14. A DNA encoding the polypeptide set forth in claim 4.

15. A manufacturing method of an optically active alcohol characterized by reacting the polypeptide set forth in claim 4 with a compound having a carbonyl group.

16. A manufacturing method of an optically active alcohol characterized by reacting a culture of the transformant set forth in claim 9 with a compound having a carbonyl group.

17. The manufacturing method set forth in claim 15, wherein (S)-3-chloro-1,2-propanediol is manufactured from 1-chloro-3-hydroxyacetone.

18. The manufacturing method set forth in claim 16, wherein (S)-3-chloro-1,2-propanediol is manufactured from 1-chloro-3-hydroxyacetone.
